(19) United States
(12) Patent Application Publication
(10) Pub. No.: US 2019/0259501 A1
(43) Pub. Date: Aug. 22, 2019

(54) METHOD FOR EVALUATION OF DISEASE RISK IN THE USER ON THE BASIS OF GENETIC DATA AND DATA ON THE COMPOSITION OF GUT MICROBIOTA

(71) Applicant: ATLAS LLC, Moscow (RU)

(72) Inventors: Sergei Vladimirovich Musienko, Korolev (RU); Andrey Valentinovich Perfilyev, Moscow (RU); Dmitrii Glebovich Alexeev, Moscow (RU); Alexander Viktorovich Tiakht, Moscow (RU); Dimitri Arkadyevich Nikogosov, Moscow (RU); Dmitrii Aleksandrovich Osipenko, Moscow (RU)

(73) Assignee: ATLAS LLC, Moscow (RU)

(21) Appl. No.: 16/186,637

(22) Filed: Nov. 12, 2018

(30) Foreign Application Priority Data
Feb. 15, 2018 (RU) 2017146240

(51) Int. Cl. G16H 50/30; G16H 70/60

(52) U.S. Cl. CPC G16H 50/30; G16H 70/60

(57) ABSTRACT

This invention relates, in general, to computer systems and methods, and, in particular, to the systems and methods for evaluation of disease risk in the user on the basis of genetic data, data on the composition of gut microbiota, filled questionnaire. The method for the assessment of disease risk in the user on the basis of genetic data and the data on the composition of gut microbiota, wherein genetic data, data on the composition of gut microbiota, genetic risk factors, external risk factors for at least one user and prevalence value of at least one disease are obtained; the adjusted odds ratio of the disease development risk in the group exposed to the risk factor to the disease development risk in the population for each risk factor is calculated for at least one user on the basis of genetic data and external risk factors; an intermediate disease risk value is calculated for the user on the basis of the disease prevalence value and adjusted odds ratio, obtained during the previous step; the relative abundance of microbial taxa in the gut microbiota of the user is calculated on the basis of the data on the composition of gut microbiota by mapping the reads to a reference database of genomes; the deviation value of the collected data on the composition of microbiota from the microbiota specific to the patients with the analyzed disease is estimated using the data on gut microbiota in the user; the final disease risk value of the user is estimated on the basis of the intermediate disease risk value and the deviation value. A technical result produced is the increase of the precision of the disease risk assessment in the user. That is achieved by the use of genetic data and/or data on the composition of gut microbiota and the filled user questionnaire.

The present application claims the benefit of Russian Patent Application RU 2017146240 filed on Dec. 27, 2017. The content of the abovementioned applicaton is incorporated by reference herein.

### FIELD OF THE INVENTION

This invention relates, in general, to computer systems and methods, and, in particular, to the systems and methods for evaluation of disease risk on the basis of genetic data and/or data on the composition of gut microbiota, filled questionnaire.

### DESCRIPTION OF RELATED ART

Disease risk is defined as the odds for a person, randomly selected from a population, to be sick with said disease. Disease development risk for a specific person is influenced by their genetic traits, features of gut microbiota, external factors, medical history, lifestyle and family history of disease.

For the purpose of calculation of disease risk (e.g. of type 2 diabetes mellitus) disease prevalence value is used as a measure of average population disease risk.

The concept of prevalence refers to already existing events, while the concept of incidence refers to novel events. Disease prevalence value is usually calculated as a ratio of total number of diagnosed cases of the disease to the population size.

Incidence is usually calculated as a ratio of the number of newly diagnosed cases of the disease in a specific period of time to the share of the population at risk of the disease. This measure shows the rate at which new cases of the disease develop in the population.

From the prior art, a U.S. Pat. No. 7,914,449B2 ‘Diagnostic support system for diabetes and storage medium’ is known, patent holder: Sysmex Corp, published on May 29, 2011. This invention provides a diagnostic system for detection of type 2 diabetes, including an input device used to input diagnostic data (including data obtained in clinical trials); a biological model comprising several parameters and representing the function of organs associated with diabetes as a numerical model; a means of predicting the values of the parameters applicable to the patient on the basis of the diagnostic data and the biological model; a means of analyzing the pathologic condition of the patient on the basis of predicted parameter values; a means of composing the diagnostic data regarding the analyzed condition; and a means of data output.

### SUMMARY OF THE INVENTION

This invention is intended to remove the shortcomings of the other inventions known in the prior art.

A technical problem solved by this invention is the assessment of disease risk in the user.

A technical result produced by the solution of the stated technical problem is the increase of the precision of the disease risk assessment in the user. That is achieved by the use of genetic data, data on the composition of gut microbiota and the filled user questionnaire.

An additional technical result produced by the solution of the problem is the personalization of recommendations on nutrition, physical activity and lifestyle for the user based on the increase of the precision of the disease risk assessment in the user.

The said technical result is obtained by the embodiment of the method for the assessment of disease risk in the user on the basis of genetic data and the data on the composition of gut microbiota, wherein genetic data, data on the composition of gut microbiota, genetic risk factors, external risk factors for at least one user and prevalence value of at least one disease are obtained; the adjusted odds ratio of the disease development risk in the group exposed to the risk factor to the disease development risk in the population for each risk factor is calculated for at least one user on the basis of genetic data and external risk factors; an intermediate disease risk value is calculated for the user on the basis of the disease prevalence value and adjusted odds ratio, obtained during the previous step; the relative abundance of microbial taxa in the gut microbiota of the user is calculated on the basis of the data on the composition of gut microbiota by mapping the reads to a reference database of genomes; the deviation value of the collected data on the composition of microbiota from the microbiota specific to the patients with the analyzed disease is estimated using the data on gut microbiota in the user; the final disease risk value of the user is estimated on the basis of the intermediate disease risk value and the deviation value.

In some embodiments of the invention average population prevalence value of the disease and/or data on the association of microbiota with the disease are obtained.

In some embodiments of the invention single-nucleotide polymorphisms (SNPs) serve as genetic risk factors.

In some embodiments of the invention external risk factors are automatically obtained from the articles that show a statistically significant association of the risk and the factor.

In some embodiments of the invention external risk values for the user are obtained from the filled user questionnaire.

In some embodiments of the invention external risk factors are modeled using epigenome-wide association studies (EWAS).

In some embodiments of the invention the data on the composition of gut microbiota are represented in FASTQ or FASTA formats.

### BRIEF DESCRIPTION OF THE DRAWINGS

Features and advantages of this invention will be apparent from the following detailed description when considered in conjunction with the drawings.

FIG. 1 is a flow chart depicting an example of a method for evaluation of disease risk in the user on the basis of genetic data and/or data on the composition of gut microbiota, filled questionnaire;

FIG. 2 is a diagram depicting the analysis of metagenomic data obtained by whole genome sequencing;

FIG. 3 is a histogram depicting the average percentage abundance of different microbial taxa in Russian and worldwide samples;

FIG. 4 depicts the average abundance of microbial genera, comprising 80% of overall coverage, by country;

FIG. 5 depicts an example of reference DNA mapping;

FIG. 6 depicts an example embodiment of a method for evaluation of disease risk in the user on the basis of genetic data and/or data on the composition of gut microbiota, filled questionnaire;

FIG. 7 depicts an embodiment where the range of possible genetic risk values is divided into 2 intervals and the range of possible values of user microbiotal deviation value is divided into 2 intervals, thus forming 4 groups.

### DETAILED DESCRIPTION OF THE INVENTION

This invention can be implemented on a computer or other data processing device in a form of an automated system or a machine-readable medium comprising instructions for performing the stated method.

The invention can be implemented in a form of a distributed computing system comprised of cloud or local servers.

In this invention, a system implies a computer system or an automated system, a computer, a numerical control, a programmable logic controller, a computerized control system and any other devices capable of performing a set sequence of specific calculations (actions, instructions).

An instruction unit implies an electronic circuit or an integrated circuit (microprocessor) that executes machine instructions (programs).

An instruction unit reads and executes machine instructions (programs) from one or more data storage devices. Data storage devices can be presented by, but are not limited to, hard disk drives (HDD), flash memory, read-only memory (RAM), solid-state drives (SSD), optical disk drives, cloud storage.

A program implies a sequence of instructions to be executed by a control unit of a computer or an instruction unit.

Described below are the terms and concepts necessary for the implementation of the invention.

Type 2 diabetes mellitus (non-insulin-dependent diabetes) is a metabolic disease characterised by chronic hyperglycemia caused by the impairment of insulin interaction with cells of tissues.

Human microbiota is a community of the microorganisms in the human body.

Genetic data is the information on DNA structure, DNA nucleotide sequence, single- and oligonucleotide polymorphisms in the DNA sequence, including all the chromosomes of a specific organism. The aspects partially determined by genetic data include, but are not limited to, morphological structure, height, development, metabolism, personality, susceptibility to diseases and malformations.

Single-nucleotide polymorphism (SNP) is the one- or several-nucleotide-long difference (nucleotides being A, T, G or C) between the genomes (or other compared sequences) of the members of the same species, or between homologous regions of homologous chromosomes.

Alleles are the different forms (values) of the same gene or the same locus (position) located in the same regions (loci) of homologous chromosomes.

DNA sequencing is the process of determination of the nucleotide sequence in a DNA molecule. It may refer to amplicon sequencing (reading the sequences of isolated DNA fragments obtained through PCR, such as a 16S rRNA gene or its fragments) or whole-genome sequencing (reading the sequences of the whole DNA present in the sample).

Locus (latin locus—place), in genetics, is the location of a particular gene or nucleotide on the genetic or cytological map of a chromosome.

Reads are data on nucleotide sequences of DNA fragments obtained using a DNA sequencer.

FASTA is a recording format used for DNA sequences.

Short reads mapping, in bioinformatics, is a method for analysis of next-generation sequencing results. It involves the identification of the positions of genes or genomes, which were most likely to produce each specific short read, in the reference database.

An array of reads is obtained as a result of DNA sequencing. Read length of modem sequencers varies from several hundreds to several thousands of nucleotides.

Taxonomy is the science concerned with the principles and practice of classification and systematization of entities with a complex hierarchical structure.

Taxon is a classification group comprised of discrete objects grouped by common properties and attributes.

16S rRNA gene is a gene present in the genomes of Bacteria and Archaea. Its nucleotide sequence is used for the taxonomic classification of these organisms.

Risk factor is a trait or a feature of a person or an influence on them that affects the odds of disease development or trauma. Risk factors can be hereditary or acquired and their influence can manifest under certain conditions.

Population (latin population) is an aggregate of the members of the same species inhabiting in the same territory for a prolonged period of time.

In medical research, as shown in reference [1], risk is defined as the odds of encountering an event in a group. Some specialists prefer to use the term ‘prevalence’ instead. The statistics of choice employed for the comparison of risks between groups of patients and/or healthy individuals are hazard ratio (HR) or relative risk (RR).

For example, if π₁ is the odds of the event in the first group and π₂ is the odds of the event in the second group, relative risk is calculated using the following formula:

$$RR = \frac{{π}_{1}}{{π}_{2}}$$

Another criterion usually used in medical literature, as shown in reference [2], is odds ratio. Odds are the ratio of the probability of the event occurring to the probability of the event not occurring. Odds ratio (OR) is the ratio of the odds of the first group of objects to the odds of the second group of objects.

A detailed description of this invention will be provided below using type 2 diabetes mellitus as an example. To a person skilled in the art it is obvious that this disease is used as an example to provide a better understanding of the invention, thus not limiting the scope of protection.

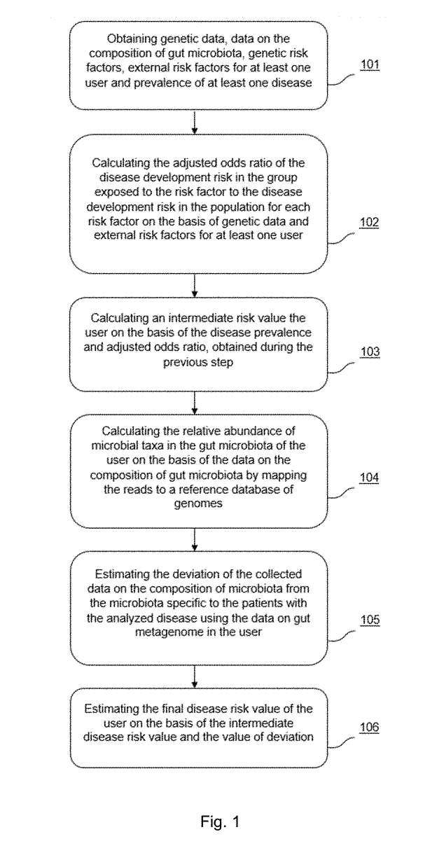

A method for evaluation of disease risk in the user can be implemented as shown in FIG. 1, comprising the following steps:

Step 101: genetic data, data on the composition of gut microbiota, genetic risk factors, external risk factors including their frequencies and their contribution represented by OR, population prevalence value of the disease and data regarding the association of gut microbiota with the disease are obtained in advance.

In some embodiments biomaterial samples from at least one user are collected. The stated data are obtained using a sampling kit comprising a sample container with a treating compound configured to receive the sample from the user sampling location. The user can deliver the samples using delivery services (e.g. postal service, courier service etc.). Additionally or alternatively, the sampling kit can be delivered using a sample collection device installed indoors or outdoors. In some embodiments the sampling kit can be delivered to a medical laboratory technician or other staff at the clinic or other medical institution. Additionally or alternatively, the sampling kit can be delivered using any other suitable method.

Preferably, the sampling kit should facilitate non-invasive collection of user samples. In some embodiments, the methods for non-invasive collection of human samples can use any or several of the following options: a permeable substrate (e.g. a tampon suitable for swabbing body surfaces, toilet paper, a sponge etc.), a container (e.g. a flask, a tube, a bag etc.), configured to receive the samples obtained from the user's body region and any other suitable sample (saliva, feces, urine etc.). In the specific example, samples can be collected non-invasively from one or several organs such as the nose, skin, genitalia, oral cavity and intestines (for example, using a tampon and a flask). Additionally or alternatively, the sampling kit may be used to facilitate semi-invasive or invasive sample collection. In some embodiments, the methods for invasive collection of samples can use, for example, a needle, a syringe, biopsy forceps, a trephine and any other instrument suitable for the invasive or semi-invasive collection of samples. In the specific examples, user samples can comprise one or several blood samples, plasma/serum samples (e.g. for the extraction of cell-free DNA) and tissue samples. Additionally, after the sample is placed in the sampling kit, it can be treated with a special solution or frozen.

Input samples can be represented by samples (saliva, urine, feces, blood) that can be treated in, for example, a laboratory, and which are later used to obtain genetic data and data on the composition of gut microbiota using genotyping or sequencing, accordingly.

In some embodiments, additional data used for the calculation of the development of type 2 diabetes mellitus in the user are obtained from the wearable sensors (e.g. PDA sensors, mobile phone sensors, wearable biometric sensors etc.). The data may regard the user's physical activity or physical interactions with the user (e.g. data obtained by the accelerometer and the gyroscope of the user's mobile phone or PDA), environmental data (e.g. data on temperature, altitude, climate, lighting etc.), nutritional data (e.g. data obtained from the registration entries of consumed food, spectrophotometric data etc.), biometric data (e.g. data obtained by the sensors of the user's PDA), location data (e.g. data obtained by GPS sensors), diagnostic data or any other suitable data. Additionally or alternatively, further data can be obtained from medical records and/or clinical findings of the user (users). In some embodiments, additional data can be obtained from a single or several electronic health records (EHRs).

Afterwards, data on the genotypes of single-nucleotide polymorphisms (SNPs) and DNA reads of user's bacteria are obtained from the samples using genotyping and sequencing.

Additionally, average disease prevalence value P₀, genetic risk factors and external risk factors are obtained for the disease (e.g. type 2 diabetes mellitus).

Average disease prevalence value P₀ shows how widespread the disease (e.g. type 2 diabetes mellitus) is in the population. It is obtained from articles or prevalence registers, where samples are composed of ethnically homogenous (e.g. Europeans only) people at a wide range of ages and both sexes are represented approximately equally.

Average disease prevalence value P₀ can be obtained automatically on request (e.g. to the API of the web platform comprising a set of articles) or by syntax analysis (parsing) of data collected by the National Center for Health Statistics and/or by Centers for Disease Control and Prevention, SIGMA T2D Consortium (Slim Initiative in Genomic Medicine for the Americas) etc., not limited to the mentioned sources. Several companies, scientific teams and research institutes determine the average disease prevalence value by dividing the overall number of both newly diagnosed cases and previously diagnosed cases that resulted in a second visit to the doctor by the population figure for a certain country, group, company etc. In some embodiments, data on a certain period of time (e.g. year 2007 or year 2017) can be used.

For example, the average disease prevalence value P₀ and the percentage of diagnosed and undiagnosed cases of type 2 diabetes mellitus in adults years old is presented in Table 1 (CI stands for confidence interval).

TABLE 1

| | Overall | | Percentage | | Percentage | |
|---|---|---|---|---|---|---|
| | percentage | | of males | | of females | |
| Trait | (95% CI) | | (95% CI) | | (95% CI) | |
| Race/ethnicity | | | | | | |
| American Indians/ | 15.1 | (15.0-15.2) | 14.9 | (14.8-15.0) | 15.3 | (15.2-15.5) |
| Indigenous Alaskans | | | | | | |
| Asian | 8.0 | (7.3-8.9) | 9.0 | (7.6-10.5) | 7.3 | (6.4-8.3) |
| Black non-Hispanic | 12.7 | (12.1-13.4) | 12.2 | (11.3-13.1) | 13.2 | (12.4-14.0) |
| Hispanic | 12.1 | (11.4-12.7) | 12.6 | (11.6-13.5) | 11.7 | (10.9-12.5) |
| White non-Hispanic | 7.4 | (7.2-7.6) | 8.1 | (7.8-8.5) | 6.8 | (6.5-7.1) |
| Education | | | | | | |
| Undergraduate or | 12.6 | (11.9-13.2) | 12.2 | (11.3-13.1) | 13.0 | (12.2-13.9) |
| lower | | | | | | |
| Graduate | 9.5 | (9.1-10.0) | 10.1 | (9.5-10.8) | 9.2 | (8.6-9.8) |
| Postgraduate or | 7.2 | (7.0-7.5) | 7.9 | (7.5-8.3) | 6.6 | (6.3-6.9) |
| higher | | | | | | |

Prevalence value P₀ can depend on the level of income in the country and may change with every passing year both increasing and decreasing.

The overall number of cases of the disease in a country, on a continent, in a city, in a company, by sex, by age or by any other criterion, needed to calculate the disease prevalence value, can be obtained at a specific point in time as well as throughout a period of time or as the number of individuals diagnosed with the disease throughout their lifetime.

Single-nucleotide polymorphisms (SNPs) can be used as risk factors. Data on the contribution of SNPs to the overall disease risk are obtained from genome-wide association studies (GWAS) with preference to GWAS meta-analyses. The search for the data employs, but is not limited by, GWAS aggregators (e.g. GWAS Catalog, GWAS Central) as well as, for example, PubMed, which is a database of medical and biological articles.

For every genetic risk factor (SNP), the following information is used:
SNP identificator (e.g. rs5749482);
the locus to which the SNP belongs (e.g. TIMP3);
reference allele (the SNP variant from the reference genome, e.g. C) and risk allele (the mutant variant or the variant of the SNP different from the reference for the population, e.g. G);
risk value (OR, RR or HR) associated with the risk allele: that is obtained either from the replication stage of the GWAS or from the combined discovery and replication data. The value of OOR can be equal to 1.31;
p-value: only the SNPs with a p-value ≤5*10⁻⁸ are used. For example, it can be equal to 2.00E−26.

For example, the genetic risk factors for type 2 diabetes mellitus are the SNPs from two loci close to ARL15 and RREB1 genes. They are strongly associated with the management of insulin and glucose levels in the body, which are the two key features of type 2 diabetes mellitus.

An SNP located in the PTEN tumor growth suppressor gene, which regulates the insulin sensitivity of the tissues, can be a genetic risk factor.

Every genetic risk factor has a frequency, which is a non-negative numerical value. Frequency is calculated per SNP allele. For example, SNP rs334 has 4 allelic variants: A, T, G and C. The frequency of T allele is 0.0274 or 2.74%.

In some embodiments, frequency is presented as a ratio or a percentage, and is always a rational number. For this purpose the ratio cannot exceed 1, and the percentage cannot exceed 100.

The determination of allele frequencies is well known from the prior art. For n people, each of whom was genotyped for a single SNP, the values for three possible SNP genotypes (A/A, A/B and B/B) can be obtained. The frequency of A allele would therefore be calculated using the following formula: P(A)=(2× N(A/A)+N(A/B))/2n. The frequency of B allele would be calculated as such: P(B)=1−P(A). The algorithm may be modified by the addition of a quality control step which checks whether the genotype distribution fits the Hardy-Weinberg equilibrium.

For example, SNP rs10012946 has three genotypes represented in the following number of people:

| | |
|---|---|
| C/C | 359 |
| C/T | 449 |
| T/T | 159 |

Therefore, allele frequency is calculated using the formula as such: T=2*T/T+T/C)/2*N=(2*159+449)/(2*967)=0.3965873837.

C allele frequency=1−T=1−0.3965873837=0.6034126163.

The list of external risk factors for the disease is at first obtained from a systematic review for a disease (e.g. type 2 diabetes mellitus). Afterwards, Internet or local storage drives are automatically searched for the original article showing a statistically significant association between the risk and the factor. Search and identification of associations are performed using a set of libraries, frameworks and packages for symbolic and statistical analysis of natural languages and speech processing and are based on the names of external risk factors (e.g. risk factors, prevention, smoking, physical activity, nutrition for the English language). These tools allow to perform sentence identification, tokenization, part of speech tagging, token recognition, lemmatization, coreference resolution. For the association to be considered statistically significant, its adjusted p-value should be lower than 0.05 and the confidence interval of its risk value (OR, RR or HR) should not contain 1.

A statistically significant association between certain external risk factors and disease risk (e.g. type 2 diabetes mellitus) is presented in Table 2, shown below. The strength of the association is represented as odds ratio (OR), the statistical significance of the association is represented as confidence interval (95% CI) of the OR and as a p-value.

TABLE 2

| Trait | OR | 95% CI | p-value |
|---|---|---|---|
| High-calorie diet | 0.76 | 0.39-1.47 | 0.20 |
| Nutritional iron intake | 0.39 | 0.19-0.79 | 0.01 |
| Nutritional vitamin A | 1.51 | 0.78-2.91 | 0.04 |
| intake | | | |
| Intake of dietary | 0.44 | 0.22-0.88 | 0.03 |
| supplements | | | |
| containing beta- | | | |
| carotene | | | |
| Intake of dietary | 1.51 | 0.78-2.91 | 0.89 |
| supplements | | | |
| containing retinol | | | |

Therefore, the main external risk factors associated with a significant increase in disease risk can be smoking, excess weight, obesity, alcohol use, infections, atmospheric pollution, radiation exposure and hereditary factors.

In some embodiments, external risk factors can have their respective weights (e.g. represented as percentages, or values from 0 to 1, or values from 0 to 100), as shown in Table 3.

TABLE 3

| | | Risk factor |
|---|---|---|
| Factor area | | respective |
| of influence | Risk factor groups | weight, % |
| Lifestyle | Smoking, alcohol use, | 49-53 |
| | unbalanced diet, distress, | |
| | harmful working conditions, | |
| | hypodynamia, poor | |
| | socioeconomic status, use of | |
| | narcotics, drug abuse, fragile | |
| | family, loneliness, low cultural | |
| | level, high urbanisation level | |
| Genetics, | Predisposition to hereditary | 18-22 |
| biology | diseases, hereditary | |
| | predisposition to | |
| | degenerative diseases | |
| Environment | Pollution of air, water or soil | 17-20 |
| | with carcinogenic and other | |
| | harmful substances, abrupt | |
| | change of atmospheric | |
| | events, increased cosmic, | |
| | ionizing, magnetic and other | |
| | types of radiation | |
| Healthcare | Ineffectiveness of preventive | 8-10 |
| | measures, low quality and | |
| | untimeliness of medical care | |

In some embodiments external risk values for the user are obtained from the filled user questionnaire.

It may, for example, comprise the following questions:
1. Specify your sex.
2. Specify your date of birth.
3. Specify your current weight in kilograms.
4. Specify your current height in centimeters.
5. Are you a smoker?

a. I am currently a smoker.

b. I used to smoke.

c. I have never smoked.
6. Does your work require you to perform physical activities of moderate intensity that result in increased heart rate and/or respiratory rate (e.g. fast walking or lifting of light weights)?

a. Yes

b. No

For example, heavy smoking or excess weight are risk factors that can influence the overall risk of type 2 diabetes mellitus development in the user.

In some embodiments external risk factors (e.g. pesticides, heavy metals, consumption of nutritional supplements) that can provoke the development of the disease (e.g. type 2 diabetes mellitus) can be modeled using epigenome-wide association studies (EWAS).

Genetic data, data on the composition of gut microbiota, genetic risk factors, external risk factors with corresponding frequencies and risk values represented as OR, population prevalence of the disease, data on the association of the composition of gut microbiota with the disease are obtained wirelessly using a stationary microcomputer unit or a mobile communication device such as a mobile phone, a smartphone or a tablet. The embodiment of the mobile communication device can provide the means of sending and receiving signals simultaneously to sending and receiving data. In particular, the information transmitted by the base station is processed by one or several processors of the system upon receipt. In general, a mobile communication device may comprise, but is not limited to, an antenna, at least one amplifier, a tuning unit, one or several emitters, a subscriber identity module (SIM) card, a transceiver, a coupling device, a low-noise amplifier, a duplexer etc. Additionally, a mobile communication device may maintain a connection to the network or other devices by wireless means. A wireless connection can employ any standard or protocol, including, but not limited to, Global System for Mobile communication (GSM), General Packet Radio Service (GPRS), code-division multiple access (CDMA), wideband code-division multiple access (WCDMA), a standard for high-speed mobile data transfer (LTE), e-mail, Short Message Service (SMS), PUSH-notifications etc.

Step 102: an adjusted ratio of the odds of disease developing in a group exposed to the risk factor to the odds of disease developing in the population is calculated for at least one user based on their genetic data and questionnaire answers.

At this step adjusted odds ratio (aOR) for every risk factor is calculated using the data processing device on the basis of user's genetic data and their questionnaire answers. Adjusted odds ratio is the ratio of the odds of type 2 diabetes mellitus developing in a group exposed to the risk factor to the odds of the disease developing in the population.

For example, an SNP rs17050272 has A as a risk allele associated with gout at the OR=1.03, and G as a reference allele.

In men, the prevalence value of gout equals 0.0397, and the genotype frequency is as follows:

### A/A 0.2332301342

### A/G 0.4963880289

### G/G 0.2703818369

Therefore, the aOR value for each genotype will be as follows:

### A/A 1.030923417

### A/G 1.000896521

### G/G 0.9717441955

The odds ratio value is similar to the relative risk value if the prevalence value is very low (prevalence value lower than 1% allows to carry the value to one decimal point).

Step 103: an intermediate disease risk value is calculated for the user on the basis of the disease prevalence value and adjusted odds ratio, obtained during the previous step;

An intermediate disease risk value for the development of the disease (e.g. type 1 diabetes mellitus) is calculated as a natural logarithm of a product of all the aOR values of the user:

$$score = ln \prod aOR ;$$

$$α = ln \frac{{P}_{0}}{1 - {P}_{0}} ;$$

wherein α is the base value for the disease and score is the user's personal component.

The value of α changes only with the change in the value of P₀, i.e., the average population disease prevalence value.

The final disease risk value based on the genetic and external risk factors is calculated using logistic regression as follows:

$$Risk = \frac{1}{1 + {e}^{- α - score}}$$

Logistic regression is used to predict the odds of an event occurring on the basis of multiple criteria. Therefore, the disease risk for type 2 diabetes mellitus is estimated by assessing the user's deviation value from the average population prevalence value (using a as the average value and score as the deviation value).

For example, disease risk for the development of type 2 diabetes mellitus for a person belonging to a British population with an average disease prevalence value of 0.063 is presented in table 4, considering their genetic and external risk factors:

TABLE 4

| SNP | Genotype | aOR |
|---|---|---|
| rs10401969 | C/T | 0.983481157 |
| rs10811661 | C/T | 0.74811823 |
| rs10830963 | G/G | 0.937018054 |
| rs10842994 | C/T | 0.866252171 |
| rs11063069 | A/A | 0.94894594 |
| | Questionnaire | |
| External risk factor | answer | |
| Eat 5 servings of fruit | no | 0.926235546 |
| weekly | | |
| Eat 5 servings of | no | 0.932016122 |
| vegetables weekly | | |
| Type 2 diabetes mellitus | yes | 0.897843805 |
| in relatives | | |
| Smoking | quit | 5.702375922 |
| | | Final risk = 2.010628467 |

Based on the data presented in Table 4, the risk for the development of type 2 diabetes mellitus in the user equals 0.11908735.

Afterwards risk distribution is assessed based on certain risk values for the development of type 2 diabetes mellitus. Risk distribution indicates what share of analyzed users corresponds to a particular risk value.

For example, the boundaries between 5 groups for type 2 diabetes mellitus in Russian women can be as follows (in ascending order):

1-2: 0.0329063148;
2-3: 0.0418203642;
3-4: 0.0612654491;
4-5: 0.0765442933;
For example, the risk value for a female user is 0.0572001.
This value is located between the second and the third boundary, placing the user in the third risk group, with the average disease risk.

For the British men the boundaries may, for example, take on the following values:

1-2: 0.0398192919;
2-3: 0.0503116186;
3-4: 0.0709393878;
4-5: 0.090999356.

That allows to rank the users by the increasing disease development risk and assign them to one of the following risk groups:
low risk (below the 10th percentile);
decreased risk (between the 10th and the 30th percentiles);
average risk (between the 30th and the 70th percentiles);
elevated risk (between the 70th and the 90th percentiles);
high risk (above the 90th percentile).

Users are assigned to the risk groups in the ascending order based on the certain disease risk values. These values are then separated into percentile segments as described above and the boundary values between the risk groups are calculated. Afterwards, the disease risk of a specific user is compared to the boundary values, and the user is assigned to one of the groups.

The boundaries are calculated on the basis of statistical data, for example, as follows: The risk values for the development of a disease (e.g. Alzheimer's disease) are calculated for real users. They are sorted in an ascending order and percentile boundary values are obtained as described above. For Alzheimer's disease in women, the boundary values are as follows:

0.04515797;
0.06140678;
0.07983051;
0.11074957.

The intermediate disease risk value is then adjusted on the basis of the data on the composition of gut microbiota in the user.

It is known from the prior art that every disease is associated with specific biomarker traits. According to a study comparing the composition of gut microbiota of type 2 diabetes mellitus patients and healthy controls, type 2 diabetes mellitus is associated with a predominance of Bacteroides bacteria and with a decrease in the population numbers of Prevotella bacteria. Bifidobacterium spp. and Bacteroides vulgatus were less represented and Clostridium leptum were better represented in the members of the disease group. The list of the biomarkers is different for the members of the European and the Asian populations, suggesting that lifestyle, sociocultural factors and ethnicity contribute to the risk.

The data on the composition of gut microbiota obtained by metagenome sequencing can be represented in FASTQ or FASTA formats, where each sample is represented with a single file.

The usage of 16S rRNA sequencing is preferable; however, whole genome sequencing (WGS) can be used as an alternative. The platforms that can be used for sequencing comprise, but are not limited by, Illumina/SOLEXA, Ion Torrent, SOLiD, Helicos.

During the analysis of the microbiota sample using 16S rRNA sequencing or WGS, each read is assigned to a known bacterial organism. That allows to perform a semiquantitative taxonomic analysis of data and calculate shares or percentage values for the sample.

Taxonomic analysis of metagenomic samples can be performed by, but is not limited to, mapping the reads to a nonredundant reference database of representative genomes and/or genes of microorganisms.

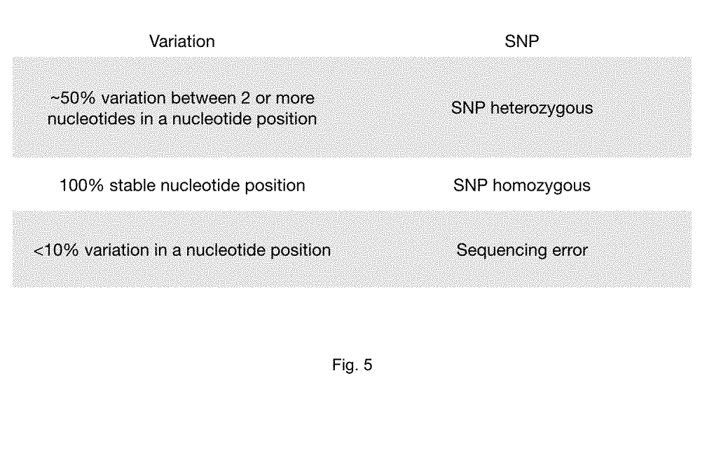

As shown in FIG. 5, a reference genome is a DNA sequence in a digital form, composed as a generic representative sample of a genetic code of a certain species.

Coverage depth is adjusted for several parameters: the overall quantity of nucleotides mapped to the reference database and the length of the genome. The sums of the adjusted values of coverage depth are calculated for each genus. The resulting values, called sample abundance vectors, are carried into the percentage of microorganisms in the sample and are used for further analysis.

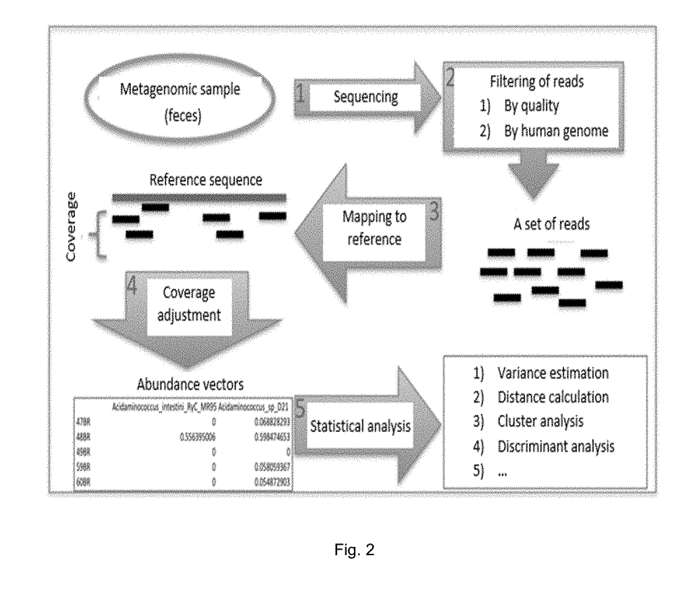

After a set of 16S rRNA metagenomic data is processed, a relative abundance table is generated as shown in FIG. 2. That table presents the number of reads corresponding to each operational taxonomic unit (OTU) from the database by sample.

In some representations, the relative metagenome abundance values are normalized (FIG. 2, step 4). To perform the normalization, the overall number of reads that were successfully mapped to the reference database is calculated for each sample. The normalized abundance value for each taxon is calculated as the ratio of the number of reads assigned to the taxon obtained from the sample to the overall number of successfully mapped reads, multiplied by 100%. The calculated normalized abundance values are then composed into an normalized abundance table that presents the percentages of reads for each taxon present in the database by sample.

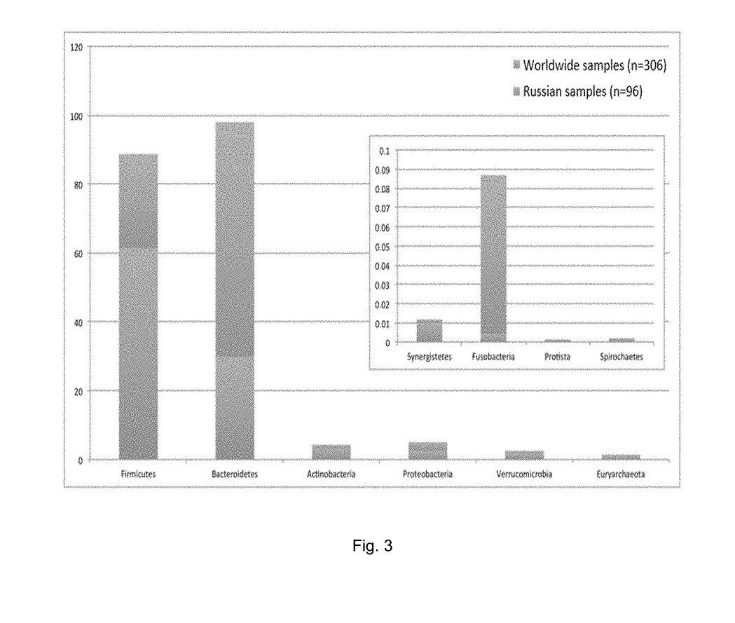

The underrepresented taxons are then filtered (FIG. 3, step 2). Filtering can be done, but is not limited by, the following criteria: only the species with the abundance of more than 0.2% of the total abundance in no less than 10% of the samples are used.

The table of normalized abundance of bacterial reads can comprise data on various taxonomic ranks up to the rank of genus. In that case, the sums of the relative sample abundance values are calculated by genus.

Overall, microbiota samples obtained from Russian and worldwide populations is primarily comprised by microbes of Bacteroidetes and Firmicutes phyli (FIG. 3).

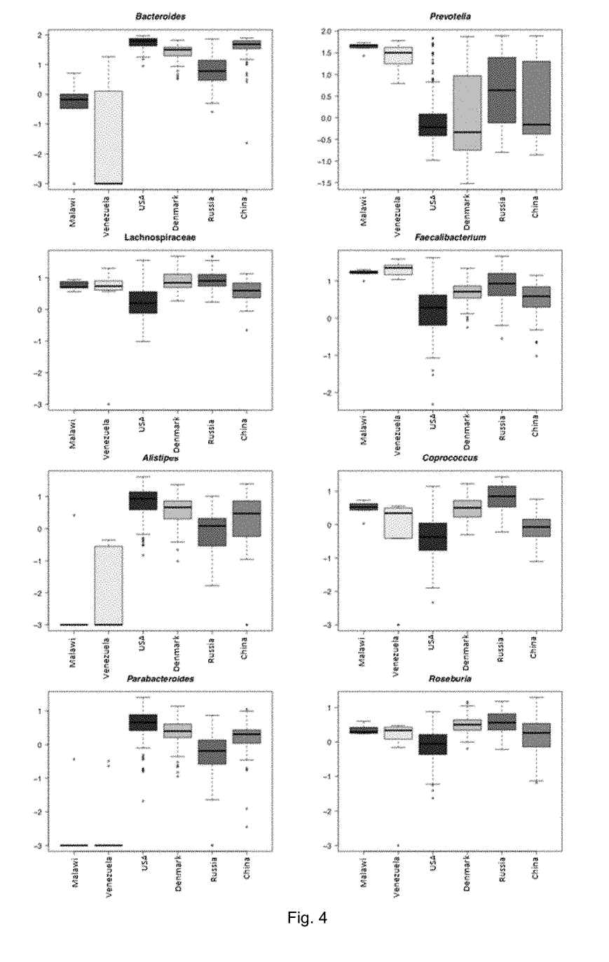

The microorganisms most represented in the samples belong to Bacteroides, Prevotella, Faecalibacterium, Alistipes, Coprococcus, Parabacteroides and Roseburia genera and to the Lachnospiraceae family. Altogether, they account for 80% of overall microbial abundance. The logarithmic representation of relative abundance values by geographic area, compared to the data obtained from earlier studies on gut microbiota in different countries, is presented on FIG. 4.

A sample fragment of Table 5 presents the percentage relative abundance of several bacterial genera (columns) in several samples (rows).

| | Acidaminococcus | Adlercreutzia | Akkermansia | Alistipes | Anaerostipes | Anaerotruncus | Bacteroides | Barnesiella | Bifidobacterium |
|---|---|---|---|---|---|---|---|---|---|
| S001 | 0.042 | 0.039 | 0.066 | 2.968 | 0.914 | 0.069 | 65.26 | 0.848 | 0.615 |
| S002 | 0.072 | 0 | 9.716 | 7.245 | 0.371 | 0.361 | 27.676 | 2.559 | 0.28 |
| S003 | 0.107 | 0.085 | 0.264 | 3.171 | 0.861 | 0.229 | 8.771 | 1.219 | 2.722 |
| S004 | 0.025 | 0 | 0.009 | 1.803 | 0.954 | 0.05 | 14.921 | 0.186 | 1.494 |
| S005 | 0.035 | 0.024 | 5.811 | 2.803 | 2.772 | 0.309 | 26.272 | 2.283 | 0.324 |
| S006 | 0.06 | 0 | 0 | 0.135 | 1.619 | 0.141 | 4.663 | 0.072 | 0.868 |
| S007 | 0.03 | 0 | 0.014 | 3.016 | 0.985 | 0.093 | 49.819 | 0.554 | 0.865 |

A context, i.e. a reference database is created in advance using the data on the composition of gut microbiota obtained from the population sample. The method employed is as follows.

A set of fixed abundance percentile values (e.g. the 33rd and the 67th percentiles) are calculated for each bacterium (by genus or any other taxon, without limitation). In other words, two abundance boundaries are calculated. In one third of the population samples, the abundance of the selected bacterium will be below the lowest boundary, while in another third it will exceed the higher boundary.

In some embodiments, the results of the statistical analysis of relative abundance of a taxon in patients affected with the disease (e.g. type 2 diabetes mellitus) in comparison to the healthy individuals can be used to calculate the values of the percentile boundaries in advance. For example, the Eubacterium genus, used as a metagenomic biomarker of type 2 diabetes mellitus, has 3.7% and 6.1% as boundary values for the 33th and the 67th percentiles, respectively.

The deviation value of the collected microbiota sample from the composition of microbiota specific to type 2 diabetes mellitus patient (henceforth referred to as deviation value from patient microbiota) is calculated using a set of biomarker taxons directly or inversely associated with the disease.

An example list of microbial biomarker taxons.

| Biomarker | Association |
|---|---|
| Firmicutes; Clostridia; Clostridiales; Ruminococcaceae; | negative |
| Subdoligranulum | |
| Verrucomicrobia; Verrucomicrobiae; Verrucomicrobiales; | negative |
| Verrucomicrobiaceae; Akkermansia | |
| Proteobacteria; Gammaproteobacteria; Pasteurellales; | negative |
| Pasteurellaceae; Haemophilus | |
| Firmicutes; Bacilli; Lactobacillales; Lactobacillaceae; | negative |
| Lactobacillus | |
| Bacteroidetes; Bacteroidia; Bacteroidales; Prevotellaceae; | negative |
| Paraprevotella | |

Step 105: the deviation value of the collected data on the composition of microbiota from the microbiota specific to the patients with the analyzed disease is estimated using the data on gut metagenome in the user.

For a sample user, a threshold deviation value can be established for type 2 diabetes mellitus. This value is calculated using the following algorithm:

For a specific sample, each microorganism (e.g. bacteria) or taxon, which is a biomarker of type 2 diabetes mellitus, is assigned a value of 0, N(k) or M(k), where k is the number of a biomarker, and N(k) and M(k) are constants specific for this biomarker of type 2 diabetes mellitus, as follows:
1. The biomarkers not represented in the sample are assigned a value of 0.
2. The biomarkers with an abundance above the lowest and below the highest percentile boundaries are assigned a value of 0.
3. The taxons not associated with the disease according to the data on the biomarkers of type 2 diabetes mellitus are assigned a value of 0.
4. The biomarkers with an abundance above the highest percentile boundary that are directly associated with the disease according to the table showing the association of biomarkers with type 2 diabetes mellitus are assigned a value of −M(k).
5. The biomarkers with an abundance below the lowest percentile boundary that are directly associated with the disease according to the table showing the association of biomarkers with type 2 diabetes mellitus are assigned a value of N(k).
6. The biomarkers with an abundance above the highest percentile boundary that are inversely associated with the disease according to the table showing the association of biomarkers with type 2 diabetes mellitus are assigned a value of 1.
7. The biomarkers with an abundance below the lowest percentile boundary that are inversely associated with the disease according to the table showing the association of biomarkers with type 2 diabetes mellitus are assigned a value of −1.

In this example, the abundance of Eubacterium genus is 2%. This genus is a biomarker of type 2 diabetes mellitus inversely associated with the disease, and its abundance is below the lowest percentile boundary (the lowest percentile boundary for Eubacterium is 3.7%). Therefore, a value of −1 is assigned.

In some approximate embodiments N(k)=M(k)=1 for all biomarkers (k=1, . . . ).

The deviation value from patient microbiota assigned to the sample for a specific disease is equal to the sum of the values assigned to the biomarkers on the previous step. For example, Eubacterium genus was assigned a value of −1, and Akkermansia genus was assigned a value of 0. If there were no additional biomarkers of type 2 diabetes mellitus, the deviation value would be equal to −1. In some embodiments, other formulas may be used to summarize the contribution of various biomarkers.

The user deviation value is then ranked using the following algorithm:
1. The lowest percentile boundary of deviation value from type 2 diabetes calculated using the context is taken as 0;
2. The highest percentile boundary of deviation value from type 2 diabetes calculated using the context is taken as 10;
3. The user deviation value is proportionally adjusted to the new scale.

The calculated value is the measure of deviation value from the patient-specific microbiota assessed by the data on the composition of gut microbiota in the user.

In some embodiments of the invention, other percentiles can be used. Additionally, each taxon can have its individual weight different from 1, −1 and 0, which is a composite of its estimated association with the trait and its abundance in the sample.

Step 106: the final disease risk group of the user is estimated on the basis of the intermediate disease risk value and the deviation value of user's microbiota from the microbiota specific to the patients with the analyzed disease.

At this step the final disease risk group of the user is estimated on the basis of the intermediate disease risk and the deviation value of user's microbiota from the microbiota specific to the patients.

The disease risk groups calculated using genetic data can be modified according to the data on the composition of gut microbiota as follows:

The risk group values associated with certain deviation values are listed below:
0-5: the disease risk group value calculated using genetic data is increased by 1, up to 5;
6-7: the risk group value is unmodified;
8-10: the disease risk group value calculated using genetic data is decreased by 1, down to 1;

If no genetic data are available, risk group can be estimated using the following concordance table:

| Microbiotic deviation value | Disease risk group |
|---|---|
| 0-3 | 5 |
| 4-5 | 4 |
| 6-7 | 3 |
| 8-9 | 2 |
| 10 | 1 |

The method for disease risk assessment is not limited by the described embodiments. Other score calculation systems may be used, as well as linear models of the association of disease risk with the genetic data and microbiota based on the data obtained from prospective studies confirming the associations.

The method for final disease risk assessment is not limited by the described embodiments and may include known associations between genetic data, external risk factors and the composition of microbiota.

In some embodiments, these associations can be estimated by calculating correlation or covariance between the genetic risk factors and the relative abundance of microbial taxa in the gut microbiota of the user.

In some embodiments, associations between parameters characteristic of the composition of gut microbiota other than microbial taxa can be analyzed, e.g. microbial genes, gene groups, metabolic pathways and alpha diversity.

These associations can be obtained from studies performed either on patients affected by the disease or any other metabolic disorder or on healthy volunteers [4].

In some embodiments, estimates of association strength can be used to calculate the weighted sum of genetic and microbiotic disease risks.

In some embodiments, the values of the weighting coefficients can be calculated according to the following principle: the higher the correlation between the abundance of the microorganism and the set of genetic risk factors for the disease, the higher the weighted coefficient for the microorganism.

In some embodiments, integral assessment that takes the known covariance between genetic risk factors, microbiotic abundance and disease development into account can be used to calculate the final risk value. For that the specific biological pathways underlying the association between the composition of microbiota, external risk factors, genetics and disease risk must be known, and it should be possible to assess the association between the abundance of the biomarker microorganism and the development of the disease [5].

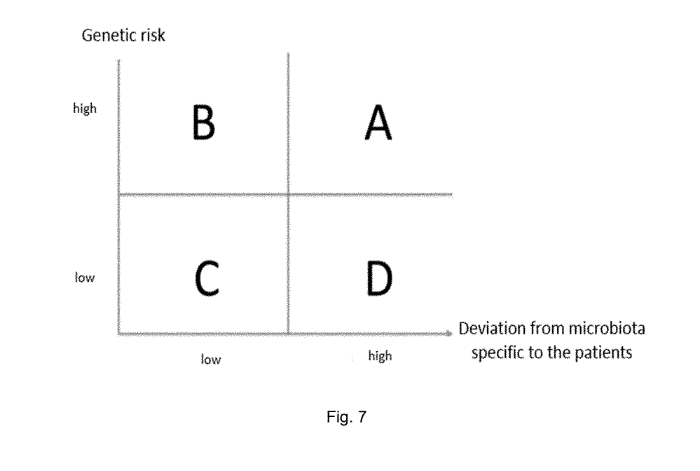

In some embodiments, risk groups may be defined as follows: both the range of possible genetic risk values and the range of possible values of user microbiotic deviation value is divided into a limited number of intervals. Each of the resulting minimal value rectangles corresponds to one risk group. It is not necessary for the groups to be sorted by ascending or descending risk. For example, 4 groups would be formed if an embodiment inferred the division of the range of possible genetic risk values into 2 intervals and of the range of possible values of user microbiotic deviation value into 2 intervals. These groups correspond to the rectangles marked A, B, C, D on FIG. 7. A person is assigned to one of the groups based on the values of these two criteria.

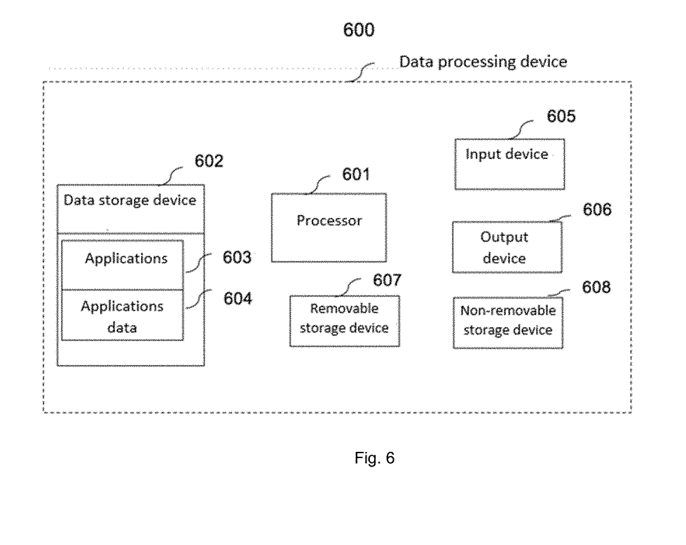

This invention can be implemented via a system for disease risk assessment in the user based on their genetic data and data on the composition of their gut microbiota. A model embodiment comprises a data processing device 600. The data processing device 600 can be configured as a client, server, mobile device or any other computer that interacts with the data in a shared network workspace. Depending on the embodiment, all the steps of the invention may be performed using one data processing device or using several data processing devices, each of which would perform several specific steps. In the basic configuration data processing device 600 is usually composed of at least one processor 601 and data storage device 602. Depending on the specifications and type of the computer, data storage device 602, which constitutes system memory, may be volatile (e.g. random-access memory, RAM), non-volatile (e.g. read-only memory, ROM) or may be presented by a combination of both types. Data storage device 602 usually comprises one or more applications 603 comprising instructions that implement the method for the assessment of disease risk in the user on the basis of genetic data and the data on the composition of gut microbiota, and may comprise the data 604 of the stated applications. A data processing device 600 can comprise additional features or capabilities. For example, a data processing device 600 can comprise additional removable and non-removable data storage devices (e.g. floppy disks, optical data disks or tape). These additional storage options are represented on FIG. 6 by a removable data storage device 607 and a non-removable data storage device 608. Computer data storage devices may comprise volatile and non-volatile, removable and non-removable data storage devices in any embodiment and using any data storage technology such as machine-readable instructions, data structures, software components or other data. Data storage device 602, removable data storage device 607 and non-removable data storage device 608 are examples of computer data storage devices. Computer data storage devices may be represented, but are not limited, by random-access memory (RAM), read-only memory (ROM), electrically erasable programmable read-only memory (EEPROM), flash-memory or memory using other technologies, compact disk read-only memory (CD-ROM), digital versatile disks (DVD) or other optical data storage devices, magnetic cassettes, magnetic tape, magnetic disks or other magnetic data storage devices or any other medium that can be used for data storage and that can be accessed by the data processing device 600. Any computer data storage device may be integrated into the data processing device 600. Data processing device 600 may additionally comprise an input device or devices 605 (e.g. a keyboard, a mouse, a stylus, a voice input device, a touch input device etc.). It may also comprise an output device or devices 606 (e.g. a display, a speaker, a printer etc.).

A data processing device 600 should comprise communication ports that would allow the device to connect to other computers (e.g. through a network). The term ‘network’ encompasses local and global networks as well as other large scalable networks that include, but are not limited by, corporate networks and extranet. A communications linkage is an example of a communication medium. Usually a communication medium may be implemented using machine-readable instructions, data structures, software components or other data carried via a modulated data signal such as a carrier wave or other device and encompasses any medium for the delivery of information. Communication mediums may be presented, but are not limited, by wiled mediums, such as wired networks or direct wired connections, and wireless mediums, such as sonic, radio, infrared and other wireless environments.

This detailed description comprises several embodiments, which are not restrictive or exhaustive. To a person skilled in the art it should be obvious that whole or partial substitutions, modifications or combinations of the presented embodiments can be reproduced without departing from the scope of the invention. It is, therefore, implied and understood that the current description of the invention comprises additional embodiments not overtly described. These embodiments may be produced by, for example, combining, modifying or transforming any steps, components, elements, qualities, aspects, specifications, limitations etc. of the mentioned embodiments, which are not restrictive.

### CITED LITERATURE

1. Stare J., Maucort-Boulch D. Odds Ratio, Hazard Ratio and Relative Risk//Metodoloski Zvezki. —2016. —T. 13. —Ng. 1. —C. 59.
2. Bland J. M., Altman D. G. The odds ratio//Bmj. —2000. —T. 320. —Ng. 7247. —C. 1468.
3. Qin J. et al. A metagenome-wide association study of gut microbiota in type 2 diabetes//Nature. —2012. —T. 490. —Ng. 7418. —C. 55-60.
4. Imhann F., Vich Vila A., Bonder M. J., et al. Interplay of host genetics and gut microbiota underlying the onset and clinical presentation of inflammatory bowel disease//Gut. —2018. T. 67. —C. 108-119.
5. Dudbridge F., Pashayan N., Yang J. Predictive accuracy of combined genetic and environmental risk scores//Genet Epidemiol. —2018. T. 42. —C. 4-19.

1. A method for calculating a disease development risk in a user on the basis of genetic data, data on the composition of gut microbiota and questionnaire results, comprising the steps of:
obtaining genetic data, data on the composition of gut microbiota, genetic risk factors, external risk factors for at least one user and a prevalence value of at least one disease;
calculating an adjusted odds ratio of the disease development risk in the group exposed to the risk factor to the disease development risk in the population for each risk factor on the basis of genetic data and external risk factors for at least one user;
calculating an intermediate disease risk value for said user on the basis of the disease prevalence value and adjusted odds ratio, obtained during the previous step;
calculating a relative abundance of microbial taxa in the gut microbiota of the user on the basis of the data on the composition of gut microbiota by mapping reads to a reference database of genomes;
calculating a deviation value of the collected data on the composition of microbiota from the microbiota specific to the patients with the analyzed disease using data on gut metagenome in the user;
calculating a final disease risk value for the user on the basis of the intermediate disease risk value and the deviation value.

2. The method of claim 1, wherein average population prevalence value of the disease and/or data on the association of microbiota with the disease are additionally obtained.

3. The method of claim 1, wherein single-nucleotide polymorphisms (SNP) are used as risk factors.

4. The method of claim 1, wherein external risk factors are automatically obtained from articles that show a statistically significant association between the risk and the factor.

5. The method of claim 1, wherein external risk values for the user are obtained from the filled user questionnaire

6. The method of claim 1, wherein external risk factors are modeled using epigenome-wide association studies (EWAS).

7. The method of claim 1, wherein the data on the composition of gut microbiota are represented in FASTQ or FASTA formats.
